(19) United States
(12) Patent Application Publication
(10) Pub. No.: US 2008/0008666 A1
(43) Pub. Date: Jan. 10, 2008

(54) Breath Test for Oral Malodor

(76) Inventor: Michael Phillips, Fort Lee, NJ (US)

Correspondence Address: DAY PITNEY LLP, 7 TIMES SQUARE, NEW YORK, NY, 10036-7311, US

(21) Appl. No.: 10/592,552

(22) PCT Filed: Mar. 14, 2005
(86) PCT No.: PCT/US05/08553

§ 371 Date: Aug. 15, 2007

Related U.S. Application Data
(60) Provisional application No. 60/552,566, filed on Mar. 12, 2004.
(60) Provisional application No. 60/562,742, filed on Apr. 16, 2004.

(51) Int. Cl. A61K 8/00; A61B 5/08; A61K 31/34; A61K 31/355

(52) U.S. Cl. 424/49; 514/458; 514/474; 600/532

(57) ABSTRACT

Measured of airway in samples a mammal, such as a human being, having oral malodor are analyzed for the presence of markers for the oral malodor. The mammal may subsequently be treated for this condition, and measured samples of airway breath again taken to determine the effectiveness of the treatment.

### BACKGROUND OF THE INVENTION

1. Field of the Invention

The present invention relates generally to the collection and analysis of breath samples. More specifically, the present invention relates to the measurement and analysis of volatile organic compounds (VOCs) responsible for oral malodor or halitosis, which are more commonly known as “bad breath”.

2. Description of the Prior Art

Normal breath includes both alveolar breath and airway breath. The former is that portion of the breath which has originated in the alveoli (“air sacs”) of the lungs, having been drawn there by inhalation for gaseous interchange with capillary blood. The latter, which is also known as “dead space” breath, is that portion of the breath which has originated in the bronchial tubes, the trachea, pharynx and mouth and nasal cavities, and comprises air in a given inhalation which has not reached the alveoli, and which therefore has not been involved in any gaseous interchange within the body.

Normal breath contains a large number of volatile organic compounds (VOCs), some of which occur in extremely low concentrations in the nanomolar or picomolar range. Many of these compounds originate in the capillary blood and enter the alveoli by diffusion across the pulmonary alveolar membrane. There they become part of alveolar breath. Other compounds arise in the airways themselves.

The collection and analysis of breath presents several technical difficulties, but may yield information of considerable medical interest. For example, there is evidence that the composition of alveolar breath may be altered in several disorders, including lung cancer, liver disease, inflammatory bowel disease, rheumatoid arthritis, heart transplant rejection, renal failure and schizophrenia. The chemical analysis of breath therefore provides a non-invasive diagnostic test for the diagnosis of these and other diseases.

U.S. Pat. No. 5,465,728 to Michael Phillips, the inventor named in the present application, discloses a highly portable apparatus used to collect mammalian breath for chemical analysis and as a diagnostic tool for the physician. The apparatus is designed to collect a large sample of alveolar breath, that is, the portion of alveolar breath in a series of exhalations, in order to concentrate and measure the volatile organic compounds (VOCs) occurring therein in low concentrations. The apparatus comprises a fluid reservoir container having first and second ends and a body extending between these ends so as to define an interior chamber; a breath entry portal; a breath exit portal; a sampling portal; a jacket including a heating system to maintain the temperature of the chamber to avoid condensation of the water vapor in the breath and the potential depletion of the volatile organic compounds of interest because of condensation. The apparatus further comprises a sample container for holding samples of exhaled breath; and pump means for moving selected samples of breath from the reservoir container into the sample container. The teachings of U.S. Pat. No. 5,465,728 are incorporated herein by reference.

U.S. Pat. No. 6,726,637 to the same Michael Phillips discloses an improved breath collection apparatus having a condensation unit, instead of a heated jacket preventing condensation. The condensation unit is disposed between a reservoir container and sorbent trap. Breath samples pass through the condensation unit, which removes water vapor therefrom, on their way to the sorbent trap. The removal of water vapor in this manner has been found, contrary to the expectations of those skilled in the art, to enhance the concentration of volatile organic compounds in alveolar breath in the sorbent trap. The teachings of U.S. Pat. No. 6,726,637 are also incorporated herein by reference.

Both of these breath collection apparatuses are specifically designed to sample alveolar breath. In a normal adult, the volume of a single breath at rest, also known as tidal breath, is approximately 500 ml, of which 150 ml is “dead space” breath and 350 ml is alveolar breath. Clearly, most of the volume of a single breath is alveolar breath. In both of the prior-art apparatuses described above, the breath sample is withdrawn from the reservoir container at a point close to where a subject exhales thereinto, so as to be primarily alveolar breath, the later portion of a breath exhaled by the subject, relatively uncontaminated by “dead space” breath. No provision is made for obtaining and studying samples primarily composed of “dead space” breath.

Accordingly, the present invention addresses this deficiency of the prior art by providing a method for obtaining a sample composed primarily of airway breath, and for analyzing, measuring and monitoring the volatile organic compounds (VOCs) therein responsible for oral malodor.

### SUMMARY OF THE INVENTION

In a first embodiment, the present invention is a method for monitoring the effectiveness of a treatment for oral malodor in a mammal, including a human. The method comprises the steps of collecting a first measured volume of airway breath from the mammal, and analyzing the first measured volume of airway breath for the presence of a marker for oral malodor. Subsequent to the treatment, which may be treatment of the oral cavity of the mammal with an antioxidant, but alternatively be another mode of treatment, a second measured volume of airway breath is collected from the mammal. The second measured volume of airway breath is analyzed for the presence of the marker for oral malodor, and the concentration of the marker in the second measured volume is compared to the concentration in the first measured volume to determine the effectiveness of the treatment.

In a second embodiment, the present invention is a method for treating oral malodor in a mammal, including a human. The method comprises the steps of collecting a measured volume of airway breath from the mammal, and analyzing the measured volume of airway breath for the presence of a marker for oral malodor. Where the marker is found, the method concludes with the step of treating the oral cavity of the mammal with an antioxidant to reduce the concentration of the marker in the breath.

The present invention will now be described more completely in the discussion to follow.

### DETAILED DESCRIPTION OF THE PREFERRED EMBODIMENTS

In accordance with the present invention, 200 ml of airway breath are collected in a bag. It will be recalled that the first portion of a breath exhaled by a subject is airway breath and has a volume, on average, of 150 ml. By collecting 200 ml in a bag, one will be reasonably sure to have collected all of the airway breath in a given exhalation. While the sample obtained may be to some degree contaminated with alveolar breath, this is not of any great concern, as will be made clear below.

The bag used is a modification of a commercially available bag for breath testing. It has a one-way diaphragm inlet valve, and an access port for sampling, namely, a luer-lock adapter with a plug, and is lined with polyethylene. The bag is available from Quintron, Inc.

It is important to note that the bag is used to collect airway breath, not alveolar breath from deep in the lungs, because most oral malodor originates in the upper airways. The volume of the bag, 200 ml, ensures that the sample taken will be primarily, if not completely, airway breath. For the study of oral malodor, it is necessary to analyze only a small sample of breath, such as 150 ml, because the volatile organic compounds (VOCs) responsible for oral malodor are present in high concentrations. This is almost “by definition”, because if a person has oral malodor, then other people can smell their breath VOCs, and this is only possible if they are very abundant. For this reason, it is not of great concern if the sample contains some alveolar breath since any VOCs that may be contributed to the sample from alveolar breath are in much smaller concentrations.

Once the sample is taken, the filled bag is shipped to a laboratory. There, it is heated to 38° C. to vaporize water condensed onto the inner surface of the bag from the breath sample. A 150 ml sample of the breath is then aspirated from the bag with a heated glass syringe, perhaps in two or three steps depending on the size of the syringe.

The breath sample is injected into a sorbent trap in order to capture the volatile organic compounds (VOCs). The sample is then analyzed by automated thermal desorption with gas chromatography and mass spectroscopy (ATD/GC/MS) and the VOCs therein are identified from a computer-based library of mass spectra.

Optionally, a condensation unit may be connected to the sorbent trap, and the breath sample may be injected so as to pass through the condensation unit before reaching the sorbent trap. The condensation unit may comprise a tube of metal or plastic maintained at room temperature. Suitable plastics include, but are not limited to, teflon and polycarbonate. Preferably, the tube is approximately 50 cm in length and has a 3 mm inside diameter. The condensation unit depletes the breath sample of water before it reaches the sorbent trap. It has been found that the use of a condensation unit results in improved capture of volatile organic components (VOCs) in the sorbent trap. It is believed that depletion of water from the breath sample results in reduced competition by water for binding sites in the sorbent trap, thereby increasing the capture of volatile organic components in the breath.

The residual gaseous components of the breath sample including the volatile organic components of interest, are then conveyed to the sorbent trap. The sorbent trap may be a stainless steel tube containing activated carbon. However, other sorbent materials or resins, such as Tenax, which is available from Supelco, Inc. of Bellefonte, Pa., may be used. Preferably, the sorbent trap includes 200 mg of Carbotrap C 20/40 mesh and 200 mg of Carbopack B 60/80 mesh, both of which are available from Supelco, Inc.

The volatile organic compounds captured in the sorbent trap may be assayed by sending the sorbent trap to a laboratory. Alternatively, the volatile organic compounds from the breath sample are desorbed from the sorbent trap by an automated thermal desorber.

The automated thermal desorber includes a heating unit which heats the sample to 200° C. or higher, and a secondary smaller trap containing sorbent material similar to the sorbent material in the sorbent trap. Upon heating, the volatile organic compounds are thermally desorbed and flushed by a stream of inert gas, such as helium or nitrogen, to the secondary smaller trap where the sample is captured and concentrated for subsequent assay.

An assay unit receives the volatile organic compounds which are desorbed from the secondary smaller trap by heating to 200° C. or higher with the automated thermal desorber. The assay unit may comprise one or more of a gas chromatograph, mass spectrometer, infrared spectroscope and an electronic nose detector to determine the identity and quantity of the volatile organic compounds. However, any instrument for analysis of volatile organic compounds may be used. Preferably, gas chromatography and mass spectroscopy are used, and the VOCs found in the sample are identified from a computer-based library of mass spectra.

### EXAMPLE 1

Patients with strong offensive oral malodor provided airway breath samples as described above. Upon analysis, the ten most abundant volatile organic compounds (VOCs) observed, in descending order, were: methylbenzene; 2,2-dimethyldecane; 2,2,3,3-tetramethylbutane; 2-propanone; 3-methyl-5-propylnonane; methylcyclohexane; 3-methylhexane; 2-methyl-1-propene; ethanol; and methylcyclopentane.

It is thought that these VOCs may be produced by bacterial metabolism. As such, the present invention may enable physicians to identify the VOCs responsible for oral malodor in individual patients, and to monitor the effectiveness of treatment to reduce their abundance.

### EXAMPLE 2

Another group of patients with strong offensive oral malodor provided airway breath samples as described above. Upon analysis, the ten most abundant volatile organic compounds (VOCs) observed, in descending order, were: methylbenzene; 2,2-dimethyldecane; 2,2,3,3-tetramethylbutane; 2-propanone; 3-methyl-5-propylnonane; methylcyclohexane; 3-methylhexane; 2-methyl-1-propene; ethanol; and methylcyclopentane.

### EXAMPLE 3

Additional patients with strong offensive oral malodor provided airway breath samples as described above. Upon analysis, the results obtained for the additional patients were combined with those of the patients evaluated for Example 2 above. The thirty most abundant VOCs in the oral cavity breath samples for all of the patients combined are shown in the Table 1 below, where they are ranked by relative abundance (mean value of ratio to abundance of an internal standard, such as a benzene derivative). An unexpected finding was that the majority of these VOCs (24 out of 30 or 80%) were alkanes or alkane derivatives, such as methylated alkanes. These are identified with an asterisk (*) in the table.

TABLE 1

| Breath VOC | Mean |
|---|---|
| Benzene, methyl- | 8.25 |
| *Butane, 2,2,3,3-tetramethyl- | 4.75 |
| Ethanol | 4.64 |
| *Hexane, 2,2,5-trimethyl- | 4.09 |
| 1-Propene, 2-methyl- | 4 |
| 1,3-Butadiene, 2-methyl- | 3.87 |
| *Nonane, 3-methyl-5-propyl- | 3.59 |
| *Decane, 2,2-dimethyl- | 3.42 |
| *Hexane, 3-methyl- | 3.18 |
| *Cyclopentane, methyl- | 2.94 |
| *Hexane | 2.84 |
| *Cyclohexane, methyl- | 2.63 |
| *Hexane, 2-methyl- | 2.63 |
| *Cyclohexane | 2 |
| *Pentane, 2,3-dimethyl- | 1.5 |
| *Undecane, 3-methyl- | 1.38 |
| *Butane, 2-methyl- | 1.21 |
| 2-Butanone | 1.11 |
| *Pentane, 3-methyl- | 1.01 |
| *Heptane | 0.96 |
| *Pentane, 3-ethyl-2,2-dimethyl- | 0.82 |
| *Decane, 2,2,8-trimethyl- | 0.75 |
| *Pentane, 2,3,3-trimethyl- | 0.75 |
| *Pentane, 2-methyl- | 0.75 |
| *Pentane, 2,3,4-trimethyl- | 0.72 |
| *Hexane, 2,2,4-trimethyl- | 0.57 |
| *Pentane | 0.55 |
| Acetaldehyde | 0.55 |
| *Cyclopentane, ethyl- | 0.54 |
| *Hexane, 2,2,3-trimethyl- | 0.53 |

VOCs which are alkanes or alkane derivatives, such as methylated alkanes are known to be products of oxidative stress, in which mitochondria produce excessive quantities of reactive oxygen species which leak into the cytoplasm and oxidize several biologically important molecules, including DNA, lipids, carbohydrates and proteins. Lipid peroxidation of polyunsaturated fatty acids generates peroxyl radical which decomposes to aldehydes and alkanes. The abundance of volatile alkanes and methylated alkanes in the breath varies with the intensity of oxidative stress.

Increased oxidative stress in the oral cavity of patients with oral malodor carries important clinical implications. Oral malodor is usually a consequence of infection in the oral cavity, and periodontal infection has been linked with increased oxidative stress. Periodontal disease has also been linked with an increased risk of atherosclerosis, coronary heart disease and stroke. These observations may be causally linked: it is possible that a focus of oral infection (e.g., gingivitis or periodontitis) generates increased oxidative stress, resulting in increased oxidation of LDL-cholesterol and accelerated atherosclerosis, thereby increasing the risk of coronary heart disease and stroke. We propose that oral malodor may be considerably more serious than merely being a social embarrassment—it may also be a sign of increased oxidative stress in the oral cavity and increased risk of life-threatening vascular disease.

The clinical findings of this study demonstrate that oxidative stress in the oral cavity appears to be source of oral malodor that has not been previously described. The probable pathophysiology of this process commences with chronic infection in oral tissues (e.g. gingivitis, periodontitis, dorsum of tongue). Reactive oxygen species generated in bacteria oxidize polyunsaturated fatty acids in the cell walls of bacteria and oral tissues to liberate alkanes and methylated alkanes which are malodorous.

These findings suggest a new approach to the treatment of oral malodor, by employing antioxidants to reduce the intensity of oxidative stress in the oral cavity. Antioxidants (including, but not limited to, vitamin E and/or vitamin C) may be applied to the oral cavity in various formulations, including, but not limited to, toothpaste, mouthwash or oral gels. The sustained effect of this therapy will be to reduce the intensity of oxidative stress in the oral cavity, thereby reducing the production of alkanes and methylated alkanes, and hence reducing the intensity of oral malodor.

What is claimed is:

1. A method for monitoring the effectiveness of a treatment for oral malodor in a mammal, including a human, said method comprising the steps of:
collecting a first measured volume of airway breath from the mammal;
analyzing said first measured volume of airway breath for the presence of a marker for oral malodor;
subsequent to said treatment, collecting a second measured volume of airway breath from the mammal;
analyzing said second measured volume of airway breath for the presence of said marker for oral malodor; and
comparing the concentration of said marker in said second measured volume to the concentration in said first measured volume to determine the effectiveness of said treatment.

2. A method as claimed in claim 1 wherein said marker is selected from the group consisting of methylbenzene; 2,2-dimethyldecane; 2,2,3,3-tetramethylbutane; 2-propanone; 3-methyl-5-propylnonane; methylcyclohexane; 3-methylhexane; 2-methyl-1-propene; ethanol; and methylcyclopentane.

3. A method as claimed in claim 1 wherein said marker is selected from the group consisting of methylbenzene; 2,2-dimethyldecane; 2,2,3,3-tetramethylbutane; 2-propanone; 3-methyl-5-propylnonane; methylcyclohexane; 3-methylhexane; 2-methyl-1-propene; ethanol; and methylcyclopentane.

4. A method as claimed in claim 1 wherein said marker is selected from the group consisting of methylbenzene; 2,2,3,3-tetramethylbutane; ethanol; 2,2,5-trimethylhexane; 2-methyl-1-propene; 2-methyl-1,3-butadiene; 3-methyl-5-propylnonane; 2,2-dimethyldecane; 3-methylhexane; methylcyclopentane; hexane; methylcyclohexane; 2-methylhexane; cyclohexane; 2,3-dimethylpentane; 3-methylundecane; 2-methylbutane; 2-butanone; 3-methylpentane; heptane; 3-ethyl-2,2-dimethylpentane; 2,2,8-trimethyldecane; 2,3,3-trimethylpentane; 2-methylpentane; 2,3,3-trimethylpentane; 2,3,4-trimethylpentane; 2,2,4-trimethylhexane; pentane; acetaldehyde; ethylcyclopentane; and 2,2,3-trimethylhexane.

5. A method for treating oral malodor in a mammal, including a human, said method comprising the steps of:
collecting a measured volume of airway breath from the mammal;
analyzing said measured volume of airway breath for the presence of a marker for oral malodor; and
treating the oral cavity of said mammal with an antioxidant to reduce the presence of said marker.

6. A method as claimed in claim 5 wherein said antioxidant is selected from the group consisting of vitamin C and vitamin E.

7. A method as claimed in claim 5 wherein said antioxidant is included in a toothpaste.

8. A method as claimed in claim 5 wherein said antioxidant is included in a mouthwash.

9. A method as claimed in claim 5 wherein said antioxidant is included in an oral gel.

10. A method as claimed in claim 5 wherein said marker is selected from the group consisting of methylbenzene; 2,2-dimethyldecane; 2,2,3,3-tetramethylbutane; 2-propanone; 3-methyl-5-propylnonane; methylcyclohexane; 3-methylhexane; 2-methyl-1-propene; ethanol; and methylcyclopentane.

11. A method as claimed in claim 5 wherein said marker is selected from the group consisting of methylbenzene; 2,2-dimethyldecane; 2,2,3,3-tetramethylbutane; 2-propanone; 3-methyl-5-propylnonane; methylcyclohexane; 3-methylhexane; 2-methyl-1-propene; ethanol; and methylcyclopentane.

12. A method as claimed in claim 5 wherein said marker is selected from the group consisting of methylbenzene; 2,2,3,3-tetramethylbutane; ethanol; 2,2,5-trimethylhexane; 2-methyl-1-propene; 2-methyl-1,3-butadiene; 3-methyl-5-propylnonane; 2,2-dimethyldecane; 3-methylhexane; methylcyclopentane; hexane; methylcyclohexane; 2-methylhexane; cyclohexane; 2,3-dimethylpentane; 3-methylundecane; 2-methylbutane; 2-butanone; 3-methylpentane; heptane; 3-ethyl-2,2-dimethylpentane; 2,2,8-trimethyldecane; 2,3,3-trimethylpentane; 2-methylpentane; 2,3,3-trimethylpentane; 2,3,4-trimethylpentane; 2,2,4-trimethylhexane; pentane; acetaldehyde; ethylcyclopentane; and 2,2,3-trimethylhexane.
